(19) United States
(12) Patent Application Publication
(10) Pub. No.: US 2026/0245702 A1
(43) Pub. Date: Aug. 20, 2026

(54) METHOD AND SYSTEM FOR REAL-TIME VIDEO ANALYSIS AND CLINICAL ACTION EXECUTION VIA MULTI-AGENT ORCHESTRATION

(71) Applicant: Cloudphysician Healthcare Pvt. Ltd., Bangalore (IN)

(72) Inventors: Dileep Raman, Bengaluru (IN); Dileep Unnikrishnan, Palakkad (IN); Dhruv Sud, Bengaluru (IN); Jitesh Sekar, Bengaluru (IN); Pradipkumar Maganbhai Solanki, Rajkot (IN)

(21) Appl. No.: 19/545,066

(22) Filed: Feb. 20, 2026

(30) Foreign Application Priority Data
Feb. 20, 2025 (IN) 202541014593

(51) Int. Cl. G16H 30/20; G06V 10/25; G16H 10/60; G16H 30/40; H04N 23/695

(52) U.S. Cl. CPC G16H 30/20; G06V 10/25; G16H 10/60; G16H 30/40; H04N 23/695; G06V 2201/07

(57) ABSTRACT

Disclosed herein, a method and system for real-time video analysis and clinical action execution via multi-agent orchestration. The method includes receiving a real-time video of a patient from a camera. The method includes assigning, via an AI model, an index tag to each of the plurality of frames in the real-time video. Upon receiving a query for the patient, the method includes retrieving, via the AI model, the plurality of frames and an associated plurality of index tags of the plurality of frames. The method includes determining, via the AI model, one or more of a plurality of AI agents based on the user query, the plurality of frames, and the associated plurality of index tags. The method includes generating, via the AI model, a combined response for the user query based on an agent response of each of the one or more of the plurality of AI agents.

### TECHNICAL FIELD

This disclosure generally relates to patient monitoring systems. More particularly, the disclosure relates to a method and a system for real-time video analysis and clinical action execution via multi-agent orchestration.

### BACKGROUND

Existing patient monitoring technologies such as bedside monitors, wearable sensors, and electronic medical record (EMR) integrations have enabled the collection of real-time patient data across clinical and home settings. The systems track vital signs, activity levels, and medication use, and in some cases, trigger alerts when certain thresholds are crossed. Some platforms allow data sharing with clinicians or limited patient access through portals, and a few incorporate basic rule-based decision support.

However, the existing technologies often operate in isolation and may fail in providing meaningful insights upon interaction with the patient data collected from the various sensors. Additionally, the existing technologies generate alerts that may be prone to false alarms and may lack context.

There is, therefore, a requirement of a method for dynamically monitor patient vitals and interacting with care delivery partners for remedial actions.

### SUMMARY

In one embodiment, a method for real-time video analysis and clinical action execution via multi-agent orchestration is disclosed. The method may include receiving a real-time video of a patient from a camera. The real-time video may include a plurality of frames. The method may further include assigning, via an Artificial Intelligence (AI) model, an index tag to each of the plurality of frames in the real-time video. The index tag may correspond to a textual description of an associated frame(s). Upon receiving a query for the patient, the method may further include retrieving, via the AI model, the plurality of frames and an associated plurality of index tags of the plurality of frames. The query is one of a user query received via a Graphical User Interface (GUI) or a system prompt. The method may further include determining, via the AI model, one or more of a plurality of AI agents based on the user query, the plurality of frames, and the associated plurality of index tags. Each of the plurality of AI agents may be preconfigured for executing a task. The method may further include generating, via the AI model, a combined response for the user query based on an agent response of each of the one or more of the plurality of AI agents.

In another embodiment, a system for real-time video analysis and clinical action execution via multi-agent orchestration is disclosed. In one example, the system may include a processor, and a memory communicatively coupled to the processor. The memory may store processor-executable instructions, which, on execution, may cause the processor to receive a real-time video of a patient from a camera. The real-time video may include a plurality of frames. The stored processor-executable instructions, on execution, may further cause the processor to assign, via an AI model, an index tag to each of the plurality of frames in the real-time video. The index tag may correspond to a textual description of an associated frame(s). Upon receiving a query for the patient, the stored processor-executable instructions, on execution, may further cause the processor to retrieve, via the model, the plurality of frames and an associated plurality of index tags of the plurality of frames. The query is one of a user query received via a Graphical User Interface (GUI) or a system prompt. The stored processor-executable instructions, on execution, may further cause the processor to determine, via the AI model, one or more of a plurality of AI agents based on the user query, the plurality of frames, and the associated plurality of index tags. Each of the plurality of AI agents may be preconfigured for executing a task. The stored processor-executable instructions, on execution, may further cause the processor to generate, via the AI model, a combined response for the user query based on an agent response of each of the one or more of the plurality of AI agents.

In yet another embodiment, a non-transitory computer-readable medium storing computer-executable instructions for real-time video analysis and clinical action execution via multi-agent orchestration is disclosed. In one example, the stored instructions, when executed by a processor, may cause the processor to perform operations including receiving a real-time video of a patient from a camera. The real-time video may include a plurality of frames. The operations may further include assigning, via an Artificial Intelligence (AI) model, an index tag to each of the plurality of frames in the real-time video. The index tag may correspond to a textual description of an associated frame(s). Upon receiving a query for the patient, the operations may further include retrieving, via the AI model, the plurality of frames and an associated plurality of index tags of the plurality of frames. The query is one of a user query received via a Graphical User Interface (GUI) or a system prompt. The operations may further include determining, via the AI model, one or more of a plurality of AI agents based on the user query, the plurality of frames, and the associated plurality of index tags. Each of the plurality of AI agents may be preconfigured for executing a task. The operations may further include generating, via the AI model, a combined response for the user query based on an agent response of each of the one or more of the plurality of AI agents.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of an exemplary system for real-time video analysis and clinical action execution via multi-agent orchestration, in accordance with some embodiments of the present disclosure.

FIG. 2 is a functional block diagram of an exemplary system for real-time video analysis and clinical action execution via multi-agent orchestration, in accordance with some embodiments of the present disclosure.

FIG. 3 illustrates a schematic diagram of an exemplary system for object detection from live streams for contextual data retrieval, in accordance with some embodiments of the present disclosure.

FIG. 4 is a flow diagram of an exemplary method for real-time video analysis and clinical action execution via multi-agent orchestration, in accordance with some embodiments of the present disclosure.

FIG. 5 is a flow diagram of an exemplary method for generating a combined response, in accordance with some embodiments of the present disclosure.

FIG. 6 illustrates a detailed flowchart of an exemplary method for real-time video analysis and clinical action execution via multi-agent orchestration, in accordance with some embodiments of the present disclosure.

FIG. 7 illustrates a functional block diagram of an exemplary system for real-time video analysis and clinical action execution via multi-agent orchestration, in accordance with some embodiments of the present disclosure.

FIG. 8 illustrates an exemplary GUI for real-time video analysis and clinical action execution via multi-agent orchestration, in accordance with some embodiments of the present disclosure.

FIG. 9 illustrates an exemplary GUI for real-time video analysis and clinical action execution via multi-agent orchestration, in accordance with some embodiments of the present disclosure.

FIG. 10 illustrates an exemplary GUI for real-time video analysis and clinical action execution via multi-agent orchestration, in accordance with some embodiments of the present disclosure.

FIG. 11 illustrates an exemplary GUI for real-time video analysis and clinical action execution via multi-agent orchestration, in accordance with some embodiments of the present disclosure.

FIG. 12 is a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

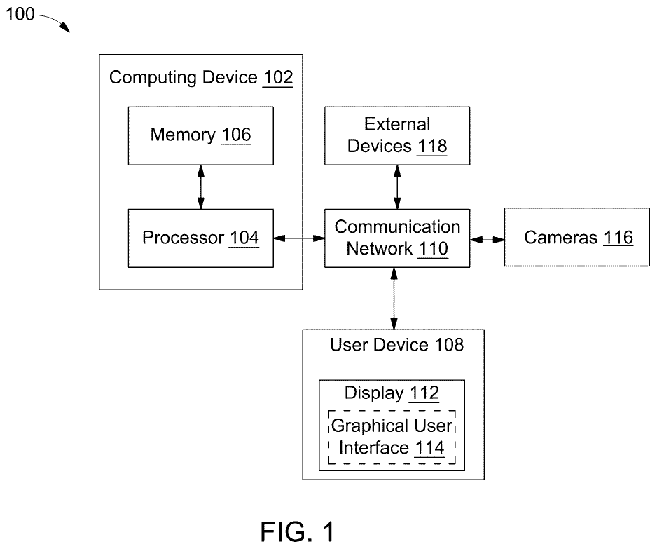

Referring now to FIG. 1, a block diagram of an exemplary system 100 for real-time video analysis and clinical action execution via multi-agent orchestration is illustrated, in accordance with some embodiments of the present disclosure. The system 100 may include a computing device 102, which, for example, may be, but is not limited to a server, a desktop, a laptop, a notebook, a netbook, a tablet, a smartphone, a mobile phone, or any other computing device. The computing device 102 may receive a real-time video and a user query for a patient as an input. Further, the computing device 102 may generate a combined response based on the response of one or more of a plurality of AI agents.

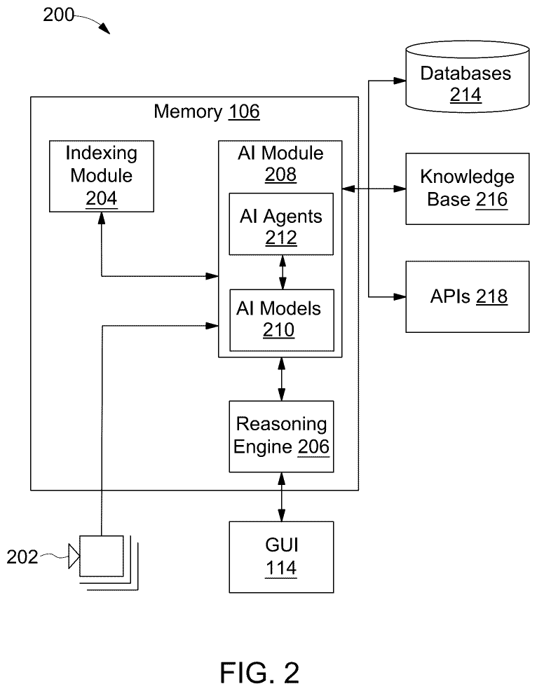

As will be described in greater detail in conjunction with FIG. 2-12, the computing device 102 may receive a real-time video of a patient from a camera. The real-time video may include a plurality of frames. Further, the computing device 102 may assign, via an AI model, an index tag to each of the plurality of frames in the real-time video. The index tag may correspond to a textual description of an associated frame(s). Upon receiving a query for the patient, retrieving, via the AI model, the plurality of frames and an associated plurality of index tags of the plurality of frames. The query is one of a user query received via a GUI or a system prompt. Further, the computing device 102 may determine, via the AI model, one or more of a plurality of AI agents based on the user query, the plurality of frames, and the associated plurality of index tags. Each of the plurality of AI agents may be preconfigured for executing a task. Further, the computing device 102 may generate via the AI model, a combined response for the user query based on an agent response of each of the one or more of the plurality of AI agents.

Further, the computing device 102 may include a processor 104 and a memory 106. In one embodiment, the computing resource may be the processor 104. The memory 106 may store instructions that, when executed by the processor 104, cause the processor 104 to monitor patients virtually, in accordance with aspects of the present disclosure. The memory 106 may also store various data (for example, a real-time video, a plurality of frames, a plurality of index tags, a combined response, a predefined threshold, a prompt, a patient summary, an EMR, a medical knowledge base and the like) that may be captured, processed, and/or required by the system 100.

The system 100 may further include a user device 108 communicatively connected to the computing device 102 over a communication network 110 for sending or receiving various data. By way of an example, the user device 108 may be, but may not be limited to, a smartphone, a tablet, a laptop, a netbook, a notebook, or any other computing device. The user device 108 may include a display 112. A user may interact with the computing device 102 via a GUI 114 accessible via the display 112. The system 100 may also include one or more cameras 116, each communicatively connected to the computing device 102 through the communication network 110. The one or more cameras 116 may be deployed in a room with a patient. The one or more cameras 116 may be configured to capture and/or record the real-time video of the patient.

Further, the system 100 may include one or more external devices 118. The computing device 102 may interact with the one or more external devices 118 over the communication network 110 for sending or receiving various data. The communication network 110, for example, may include, but may not be limited to, a Wireless Fidelity (Wi-Fi) network, a Light Fidelity (Li-Fi) network, a Local Area Network (LAN), a Wide Area Network (WAN), a Metropolitan Area Network (MAN), a satellite network, the internet, a fiber optic network, a coaxial cable network, an infrared (IR) network, a Radio Frequency (RF) network, or a combination thereof. The one or more external devices 118 may include, but may not be limited to a remote server, a laptop, a netbook, a notebook, a smartphone, a mobile phone, a tablet, or any other computing device.

Referring now to FIG. 2, a functional block diagram of an exemplary system 200 configured for real-time video analysis and clinical action execution via multi-agent orchestration is illustrated, in accordance with some embodiments of the present disclosure. FIG. 2 is explained in conjunction with FIG. 1. The system 200 may be analogous to the system 100. The system 200 may include a camera 202 (analogous to the one or more cameras 116). The system 200 may further include, within the memory 106 of the computing device 102, an indexing module 204, a reasoning engine 206, and an AI module 208. The AI module 208 may include a set of AI models 210 and a plurality of AI agents 212. By way of an example, the set of AI models 210 may include, but may not be limited to, Computer Vision (CV) models, multimodal AI models with transformer architecture, hierarchical reasoning models of transformer architecture, and the like. In some embodiments, one or more of the set of AI models 210 may be hosted on external servers. It should be noted that one or more of the plurality of AI agents 212 may be vision-based AI agents.

The system 200 may further include one or more databases 214 (for example, a patient Electronic Medical Record (EMR) database, device databases, environment databases, etc.), a medical knowledge base 216, and one or more Application Programming Interfaces (APIs) 218. It should be noted that the one or more databases 214, the knowledge base 216, and the one or more APIs 218 may be hosted on external devices (for example, external servers).

The camera 202 may capture a real-time video of a patient. The camera 202 may then send the real-time video to the AI module 208. The real-time video may include a plurality of frames. Further, the AI module 208 may generate a textual description for each of the plurality of frames using an AI model from the set of AI models 210. In an embodiment, the AI model may be one of the multimodal AI models with transformer architecture. In some embodiments, the plurality of AI agents 212 may include an indexing agent pre-configured to interact with the AI model to generate the textual descriptions for the plurality of frames. The AI module 208 may then send the corresponding textual description to the indexing module 204.

The indexing module 204 may assign an index tag to each of the plurality of frames in the real-time video. The index tag may correspond to the textual description of an associated frame generated by the AI model. The index tag and the associated frame of the real-time video may be stored in the one or more databases 214.

The system 200 may further include a user device (such as the user device 108) in communication with the computing device 102. The GUI 114 may be rendered on the display 112 of the user device 108. The GUI 114 may present the real-time video of the patient along with various patient health parameters (e.g., vitals). The GUI 114 may also provide a GUI element for a user to input a user query related to the patient. The user query may be in text, audio, or video format. Once the user provides the user query through the GUI 114, the user query is sent to the reasoning engine 206 of the computing device 102. Additionally, a system prompt may be autonomously generated and transmitted to the reasoning engine 206. For example, the system prompt may be autonomously generated by a vision-based AI agent when an anomaly is detected in a frame of the real-time video by the vision-based AI agent. As will be appreciated, the generation and transmittal of the system prompt may not require any user intervention. Hereinafter, the terms “user query” and “system prompt” are collectively referred to as “query”.

Upon receiving the query for the patient, the reasoning engine 206 may retrieve, using an AI model from the set of AI models 210, the plurality of frames and an associated plurality of index tags of the plurality of frames from the one or more databases 214. Further, the reasoning engine 206 may select one or more of the plurality of AI agents 212 to address the query. Particularly, reasoning engine 206 may determine, using an AI model from the set of AI models 210, one or more of the plurality of AI agents 212 based on the query, the plurality of frames, and the associated plurality of index tags. The AI model used for determining the one or more of the plurality of agents 212 may be one of the hierarchical reasoning models of transformer architecture. Advantageously, use of the plurality of index tags may increase the efficiency of retrieval of relevant data for the query from the one or more databases 214.

In some embodiments, the plurality of AI agents 212 may include, but may not be limited to, at least one of an indexing agent, a data retrieval agent, an EMR management agent, a camera control agent, and a communication agent. Each of the plurality of AI agents 212 may be preconfigured for executing a task. One or more of the plurality of AI agents may be vision-based AI agents. For example, the indexing agent, the patient data retrieval agent, and the camera control agent may be vision-based AI agents. Further, the reasoning engine 206 may determine an order of processing of the query through the one or more of the plurality of AI agents 212. In other words, the reasoning engine 206 may determine, using the AI model from the set of AI models 210, a sequence in which each of the one or more of the plurality of AI agents 212 may process the query. It is to be noted that the AI model used in determining the order of processing may be one of the hierarchical reasoning models of transformer architecture. Further, the reasoning engine 206 may send the query to the AI module 208.

Further, the AI module 208 may generate an agent response through each of one or more of the plurality of AI agents 212 in the determined order of processing. The one or more AI agents are selected from the plurality of AI agents 212 based on an analysis of the query and the pre-configured tasks of the plurality of AI agents 212. In other words, each of the plurality of AI agents 212 is pre-configured to perform a task using an AI model from the set of AI models 214. For example, the patient data retrieval agent may generate a patient summary based on at least one of patient data retrieved from an EMR of the patient or medical domain data retrieved from the medical knowledge base 216. The EMR management agent may execute, an order action in the EMR through a first API of the one or more APIs 218 based on the query. The order action may correspond to one of a new order addition, an order cancellation, or an order modification. The camera control agent may transmit a control instruction to the camera through a second API of the one or more APIs 218 based on the query for execution of a camera action to modify a current field of view (FoV) of the camera 202. The camera action may correspond to one of a pan action, a tilt action, or a zoom action. Thus, the computing device 102 is also configured to control the camera 202 based on the query or a system prompt. The communication agent may execute a communication action on the GUI through a third API of the one or more APIs 218 based on the query or the system prompt. The communication action may be one of a message transmission or a call initiation.

Upon receiving the agent response from the one or more of the plurality of agents 212, an AI model from the set of AI models 210 may generate a combined response based on sequential processing of the query, the plurality of frames, and the associated plurality of index tags through the one or more of the plurality of AI agents 212 in the order of processing. The AI model used for generating the combined response from individual agent responses may be a language model (such as a reasoning model of transformer architecture, or any deep learning model configured for Natural Language Processing (NLP)).

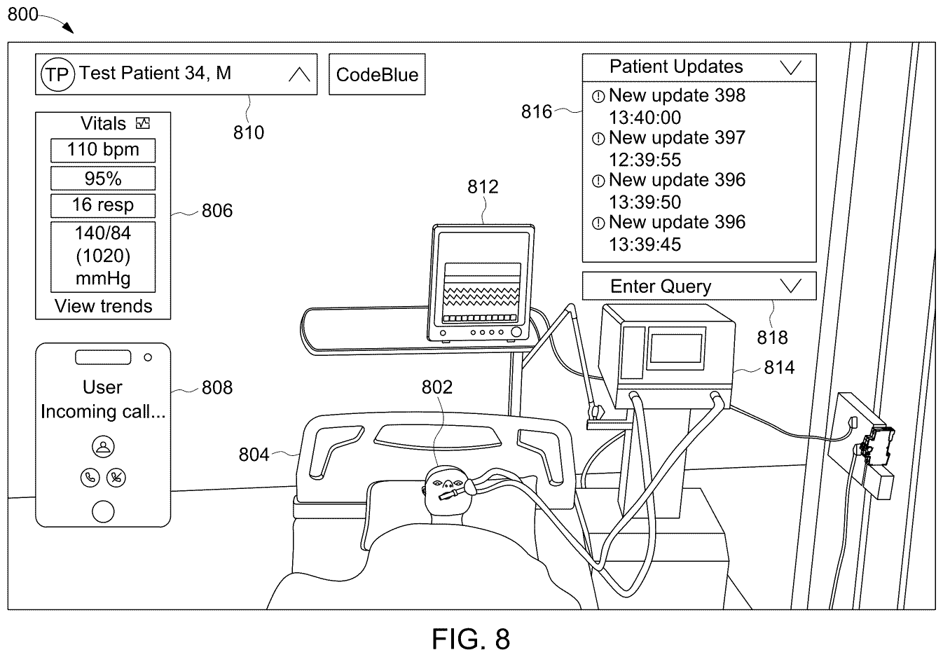

To generate an agent response, for each agent of the one or more of the plurality of AI agents 212, the AI module 208 may create, via the AI agent, a prompt based on a set of agent instructions and at least one of the query, the plurality of frames, or the associated plurality of index tags. Further, the AI agent may input the prompt to the associated AI model. Further, the AI model may generate the agent response based on the prompt. Further, to generate the combined response the language model may combine the agent response for each of the one or more of the plurality of AI agents 212 to obtain the combined response. The AI module 208 may send the combined response to the reasoning engine 206. Further, the reasoning engine 206 may render, via the GUI 114, the real-time video of the patient and the combined response to the query. The combined response may be overlaid on the real-time video of the patient in the GUI 114. This is explained in greater detail in conjunction with FIGS. 8-11.

It should be noted that all such aforementioned modules 204-208 may be represented as a single module or a combination of different modules. Further, as will be appreciated by those skilled in the art, each of the modules 204-208 may reside, in whole or in parts, on one device or multiple devices in communication with each other. In some embodiments, each of the modules 204-208 may be implemented as dedicated hardware circuit comprising custom application-specific integrated circuit (ASIC) or gate arrays, off-the-shelf semiconductors such as logic chips, transistors, or other discrete components. Each of the modules 204-208 may also be implemented in a programmable hardware device such as a field programmable gate array (FPGA), programmable array logic, programmable logic device, and so forth. Alternatively, each of the modules 204-208 may be implemented in software for execution by various types of processors (e.g., the processor 104). An identified module of executable code may, for instance, include one or more physical or logical blocks of computer instructions, which may, for instance, be organized as an object, procedure, function, or other construct. Nevertheless, the executables of an identified module or component need not be physically located together but may include disparate instructions stored in different locations which, when joined logically together, include the module, and achieve the stated purpose of the module. Indeed, a module of executable code could be a single instruction, or many instructions, and may even be distributed over several different code segments, among different applications, and across several memory devices.

As will be appreciated by one skilled in the art, a variety of processes may be employed for real-time video analysis and clinical action execution via multi-agent orchestration. In particular, as will be appreciated by those of ordinary skill in the art, control logic and/or automated routines for performing the techniques and steps described herein may be implemented by the system 100 and the associated computing device 102, either by hardware, software, or combinations of hardware and software. For example, suitable code may be accessed and executed by the one or more processors on the system 100 to perform some or all of the techniques described herein. Similarly, application specific integrated circuits (ASICs) configured to perform some, or all of the processes described herein may be included in the one or more processors on the system 100.

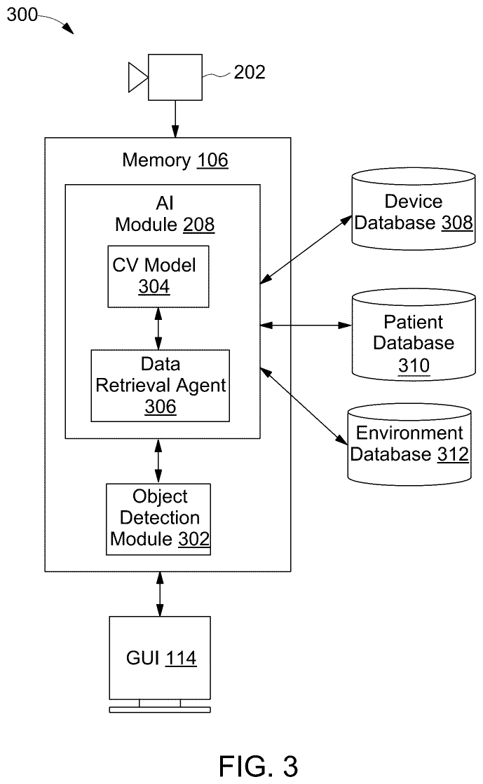

Referring now to FIG. 3, a schematic diagram of an exemplary system 300 for object detection from live streams for contextual data retrieval is illustrated, in accordance with some embodiments of the present disclosure. FIG. 3 is explained in conjunction with FIGS. 1 and 2. In an embodiment, the system 300 may be deployed in combination with the system 200. It should be noted that in such a deployment, the combination of the system 200 and the system 300 may be analogous to the system 100 of FIG. 1. The system 300 may include the camera 202 communicably coupled to the computing device 102. The computing device 102 may include, within the memory 106, the AI module 208 and an object detection module 302. The AI module 208 may include a CV model 304 and a data retrieval agent 306. It should be noted that the CV model 304 may be one of the set of AI models 210. It should also be noted that the data retrieval agent 306 may be one of the plurality of AI agents 212. Further, the system 300 may include a device database 308, a patient database 310, and an environment database 312. It should be noted that the device database 308, the patient database 310, and the environment database 312 are included in the one or more databases 214.

Further, the computing device 102 may be communicably coupled to the user device 108. The user device 108 may render the GUI 114 on the display 112. The camera 202 may capture the real-time video of the patient. Further, the object detection module 302 may receive the real-time video from the camera 202. The object detection module 302 may send the real-time video to the AI module 210. Further, the AI module 210 may identify, using the CV model 304, one or more objects around the patient in each of the plurality of frames of the real-time video. By way of an example, the CV model 304 may be based on a Convolutional Neural Network (CNN) architecture. The one or more objects may include, but are not limited to, face of the patient, body and behaviour of the patient, screens of various patient health monitoring devices (such as a cardiac monitor, a ventilator screen, etc.), tubes and bags attached to the patient (e.g., urine collection bag, IV fluid bag, etc.) or the like.

For each object of the one or more objects, the CV model 304 may determine an area of coverage of the object in the frame. When the area of coverage may be above a predefined threshold, the data retrieval agent 304 may retrieve relevant data (at least one of relevant patient data, device data, or environment data) associated with the object from one or more of the databases 214 (i.e., from one or more of the patient database 310, the device database 308, and the environment database 312). The predefined threshold may be any predetermined proportion of the frame. In one example, the predefined threshold may be 60% area of coverage of the frame. When an object is detected with the coverage of the frame above the predefined threshold, the relevant data may be retrieved by the data retrieval agent 306. The relevant data may then be rendered on the user device 304 via the GUI.

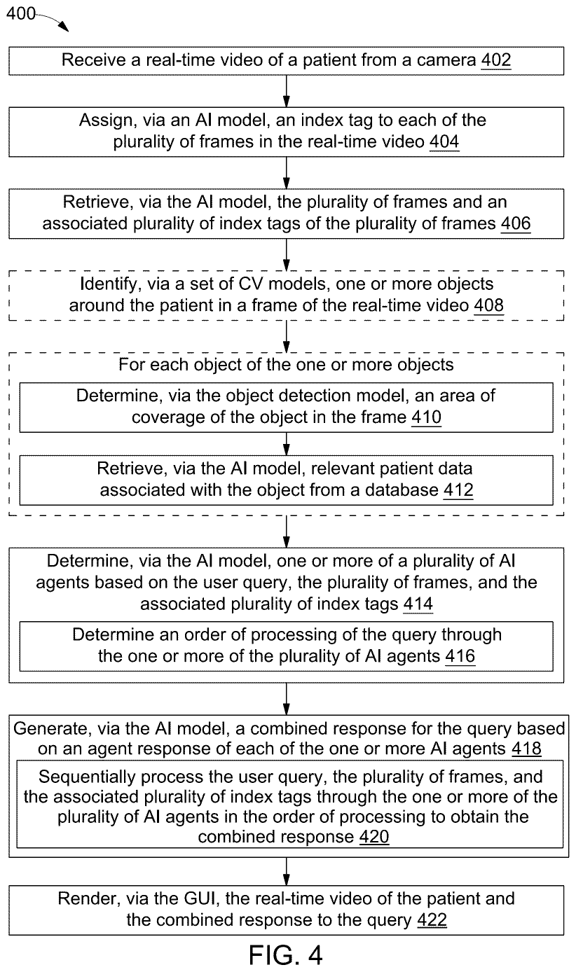

Referring now to FIG. 4, a flow diagram of an exemplary method 400 for real-time video analysis and clinical action execution via multi-agent orchestration is illustrated, in accordance with some embodiments of the present disclosure. FIG. 4 is explained in conjunction with FIGS. 1-3 The method 400 may be implemented by the computing device 102 of the system 100. The method 400 may include receiving a real-time video of a patient from a camera (such as the camera 202), at step 402. The real-time video may include a plurality of frames. Further, the method 400 may include assigning, via an AI model, an index tag to each of the plurality of frames in the real-time video, at step 404. The index tag may correspond to a textual description of an associated frame. Upon receiving a query for the patient, the method may include retrieving, via the AI model, the plurality of frames and an associated plurality of index tags of the plurality of frames, at step 406. The query is one of a user query received via a GUI (for example, the GUI 114) or a system prompt. Further, the method 400 may include identifying, via a set of CV models (such as the CV model 302), one or more objects around the patient in a frame of the real-time video, at step 408.

For each object of the one or more objects, the method 400 may include determining, via the set of CV models, an area of coverage of the object in the frame, at step 410. When the area of coverage may be above a predefined threshold, the method 400 may include retrieving, via the AI model, at least one of relevant patient data, device data, or environment data associated with the object, at step 412. Further, the method may include determining, via the AI model, one or more of a plurality of AI agents based on the user query, the plurality of frames, and the associated plurality of index tags, at step 414. The step 414 may include step 416. Each of the plurality of AI agents may be preconfigured for executing a task. The plurality of AI agents may include at least one of a patient data retrieval agent, an Electronic Medical Record (EMR) management agent, a camera control agent, and a communication agent. Further, the method 400 may include determining an order of processing of the user query through the one or more of the plurality of AI agents, at step 416. Further, the method 400 may include generating, via the AI model, a combined response for the user query based on an agent response of each of the one or more of the plurality of AI agents, at step 418. The step 418 may include step 420. Further, the method 400 may include sequentially processing the user query, the plurality of frames, and the associated plurality of index tags through the one or more of the plurality of AI agents in the order of processing to obtain the combined response. Further, the method 400 may include rendering via the GUI, the real-time video of the patient and the combined response to the user query, at step 422. The combined response may be overlaid on the real-time video of the patient in the GUI.

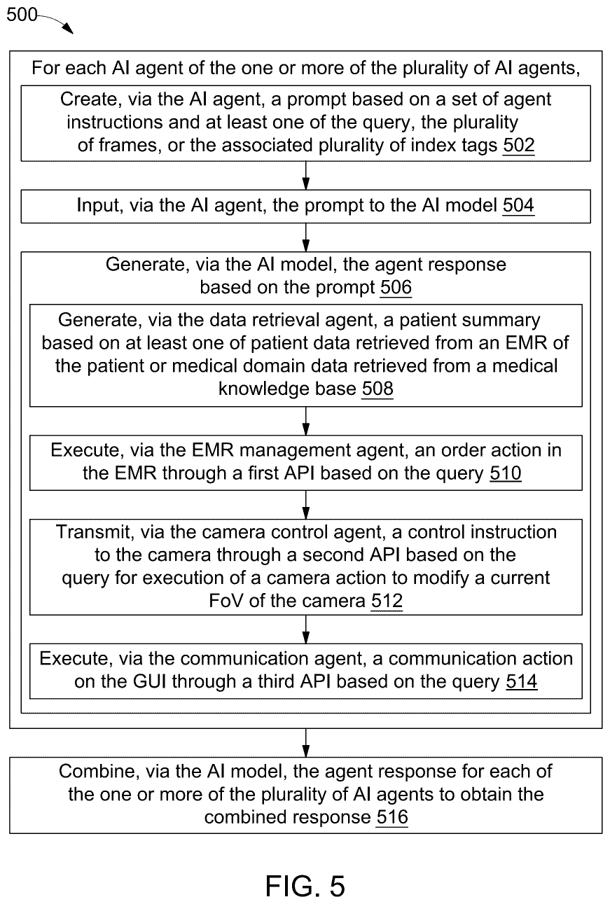

Referring now to FIG. 5, a flow diagram of an exemplary method 500 for generating a combined response is illustrated, in accordance with some embodiments of the present disclosure. FIG. 5 is explained in conjunction with FIG. 1-4. The method 500 may be implemented by the computing device 102 of the system 100. For each AI agent of the one or more of the plurality of AI agents, the method 500 may include creating, via the AI agent, a prompt based on a set of agent instructions and at least one of the user query, the plurality of frames, or the associated plurality of index tags, at step 502. The method 500 may further include inputting, via the AI agent, the prompt to the AI model, at step 504. The method 500 may further include generating, via the AI model, the agent response based on the prompt, at step 506. The agent response generation may include steps 508-514. To generate the agent response, the method 500 may include generating, via the patient data retrieval agent, a patient summary based on at least one of patient data retrieved from an EMR of the patient or medical domain data retrieved from a medical knowledge base, at step 508. Further, the method 500 may include executing, via the EMR management agent, an order action in the EMR through a first API based on the query, at step 510. The order action may correspond to one of a new order addition, an order cancellation, or an order modification.

Further, the method 500 may include transmitting, via the camera control agent, a control instruction to the camera through a second API based on the user query for execution of a camera action to modify a current FoV of the camera, at step 512. The camera action may correspond to one of a pan action, a tilt action, or a zoom action. Further, the method 500 may include executing, via the communication agent, a communication action on the GUI through a third API based on the user query, at step 514. The communication action may be one of a message transmission or a call initiation.

Further, the method 500 may include combining, via the AI model, the agent response for each of the one or more of the plurality of AI agents to obtain the combined response, at step 516.

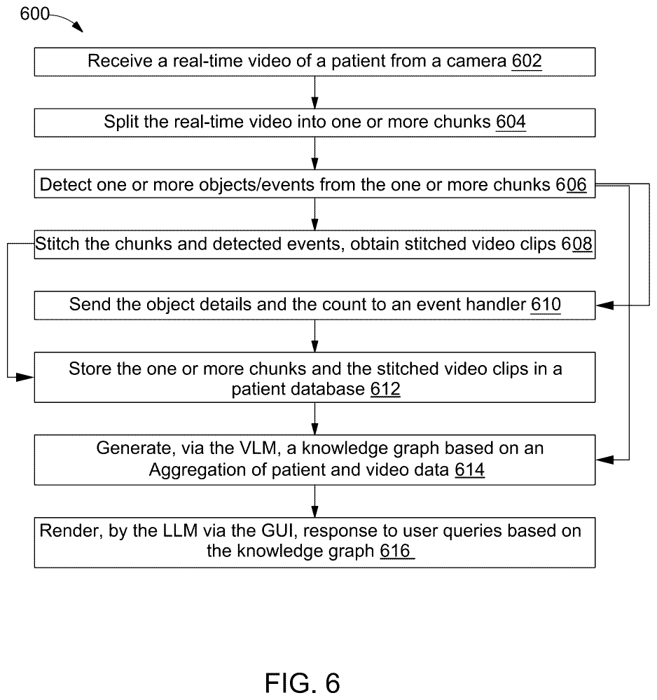

Referring now to FIG. 6, a detailed flowchart of an exemplary method 600 for real-time video analysis and clinical action execution via multi-agent orchestration is illustrated, in accordance with some embodiments of the present disclosure. FIG. 6 is explained in conjunction with FIG. 1-5. The method 600 may be implemented by the computing device 102 of the system 100. The method 600 may include receiving a real-time video of a patient from a camera (such as the camera 202), at step 602. The real-time video may include a plurality of frames. Further, the method 600 may include splitting the real-time video into one or more chunks, at step 604. A chunk may include one or more of the plurality of frames. Additionally, after step 604, the method 600 may include detecting one or more objects/events from the one or more chunks, at step 606. The object/event details and parameters associated with the objects detected may be stored in a timeseries database. For example, the parameters may include respiratory rate detected on a ventilator screen detected in a frame. In some embodiments, the timeseries database may store timestamp corresponding to the one or more objects in the real-time video. The timeseries database may help in detecting the exact time when an undesired event happened. In one example, if the heart rate may increase or decrease beyond a predefined standard value that timestamp may be determined from the timeseries database and the corresponding events triggered due to the change may be noted to determine remedial actions. The remedial actions may include but are not limited to increase or decrease sedation, administer pain medications and escalate antibiotics.

Further, the method 600 may include stitching the one or more chunks and the detected objects/events to obtain stitched video clips, at step 608. Further, the method 600 may include sending the object/event details and the parameters to an event handler, at step 610. Further, the method 600 may include storing, via the event handler, the one or more chunks and the stitched video clips in a patient database based on events, at step 612. Further, the method 600 may include sending the detected events and the one or more chunks to a multimodal model (for example, a Vision Language Model (VLM) or any other multimodal AI model with a transformer architecture). Further, the method 600 may include generating, via the VLM, a knowledge graph based on an aggregation of patient and video data, at step 614. The aggregated data may include detected events, object details, count of objects, patient's EMR data, the one or more chunks and the like. Further, the method 600 may include rendering, by the set of AI model and AI agents via the GUI, response to a user query based on the knowledge graph, at step 618. The output may be overlaid on the real-time video of the patient in the GUI.

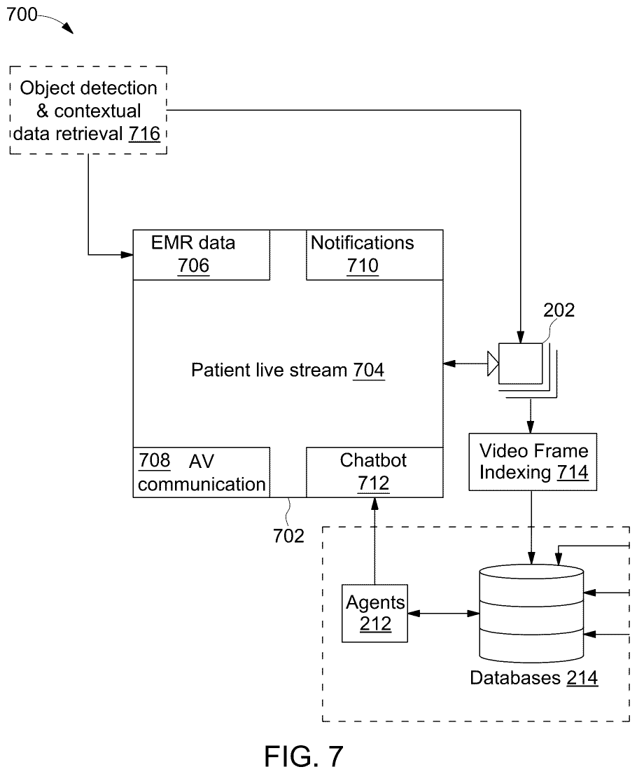

Referring now to FIG. 7, a functional block diagram of an exemplary system 700 for real-time video analysis and clinical action execution via multi-agent orchestration is illustrated, in accordance with some embodiments of the present disclosure. FIG. 7 is explained in conjunction with FIG. 1-6. The system 700 may include a camera 202. The camera 202 may be configured to capture a real-time video of the patient. Further, the system 700 may include the user device 108 (not shown). The user device 108 may render a GUI 702 (analogous to the GUI 114) on the display 112. The GUI 702 may present the real-time video as a patient live stream 704. Additionally, the GUI 702 may include auxiliary data and GUI elements overlaid upon the real-time video. For example, the auxiliary data and GUI elements may include EMR data 706 overlaid at top left corner, an AV communication element 708 overlaid at bottom left corner, notifications 710 overlaid at right corner, and a chatbot 712 overlaid at bottom right corner.

The system 700 may also include the computing device 102 (not shown) communicably coupled to the camera 202 and the user device 108. The computing device 102 may implement a video frame indexing component 714 and an object detection and contextual data retrieval component 716. It should be noted that the system 700 may be analogous to the system 200 and the system 300. It should also be noted that the implementation of the video frame indexing component 714 and the object detection and contextual data retrieval component 716 may be facilitated by equivalent modules defined in the system 200 and the system 300 in conjunction with FIG. 2 and FIG. 3, respectively.

The camera 202 may capture the patient live stream 704 and may transmit the patient live stream 704 to the object detection and contextual data retrieval component 716. The object detection and contextual data retrieval component 702 may perform object detection and contextual data retrieval functions on the incoming video stream. This has already been explained in detail in conjunction with FIG. 3.

The video frame indexing component 714 may receive the patient live stream 704 from the camera 202 and may process each frame to generate textual descriptions, assign index tags to each frame based on the generated textual descriptions, and store the indexed frames along with the associated index tags in the databases 214. The indexed frames and index tags may be subsequently retrieved by the agents 212 when processing queries (i.e., system prompts or user queries received through the chatbot 712). This has already been explained in detail in conjunction with FIG. 2.

The chatbot 712 may provide an interface for a user to input queries related to the patient. Upon receiving a user query through the chatbot 712, the computing device 102 may determine one or more of the agents 212 to process the query based on the query content, the indexed frames, and the associated index tags. The agents 212 may generate individual agent responses, which may be combined to form a combined response. The combined response may be rendered via the chatbot 712 and may be overlaid on the patient live stream 704 in the GUI 702.

The EMR data 706 may display patient health parameters retrieved from the databases 214, including vital signs, laboratory values, and medication information. The notifications 710 may display alerts and patient updates generated based on detected events in the patient live stream 704. The AV communication element 708 may enable audio and video communication between the user and other healthcare personnel or the patient, facilitating remote consultation and care coordination.

Referring now to FIG. 8, an exemplary GUI 800 for real-time video analysis and clinical action execution via multi-agent orchestration is illustrated, in accordance with some embodiments of the present disclosure. FIG. 8 is explained in conjunction with FIG. 1-7. The GUI 800 may display a real-time video feed of a patient 802 lying on a hospital bed 804 in a clinical setting. A vitals panel 806 may be positioned on the upper left corner of the GUI 800 and may display patient vital signs including heart rate, oxygen saturation, respiratory rate, and blood pressure readings. A communication interface 808 may be located on the lower left corner of the GUI 800 and may show an incoming call notification with options to accept or decline the call. A patient information bar 810 may be positioned at the top centre of the GUI 800 and may display patient identification information including initials, name, age, and gender, along with a status indicator. A patient monitor 812 may be visible in the background of the real-time video feed mounted on an adjustable arm near the hospital bed 804. A ventilator screen 814 may be positioned adjacent to the patient monitor 812 and may display ventilator-related information. A notification center 816 may be located on the upper right corner of the GUI 800 and may display a list of patient updates with timestamps. An AI chat interface 818 may be positioned below the notification center 816 and may provide an interface for multimodal interaction with a reasoning engine.

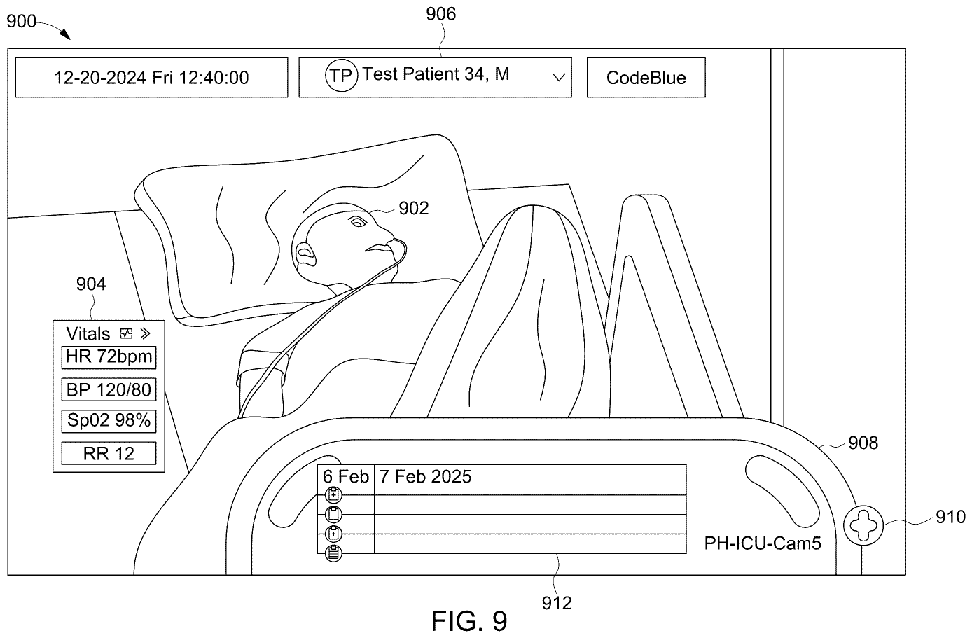

Referring now to FIG. 9, a GUI 900 for real-time video analysis and clinical action execution via multi-agent orchestration is illustrated, in accordance with some embodiments of the present disclosure. FIG. 9 is explained in conjunction with FIG. 1-8. The GUI 900 may display a real-time video 902 of a patient lying in a hospital bed as the background of the interface. A vitals panel 904 may be positioned on the left side of the GUI 900 and may display patient vital signs including heart rate, blood pressure, SpO2, and respiratory rate. A patient information display 906 may be located at the top center of the GUI 900 and may show patient identification information including the patient name and a status indicator. Bedside equipment 908 may be visible on the right side of the real-time video 902 and may include items (such as beverage containers and medical supplies on a bedside table). An AI interaction interface 910 may be positioned in the lower right portion of the GUI 900 and may provide a mechanism for multimodal interaction with a reasoning engine. A timeline interface 912 may be displayed at the bottom of the GUI 900 and may show a chronological view of patient events with date markers and event indicators, allowing navigation through recorded patient data and video streams.

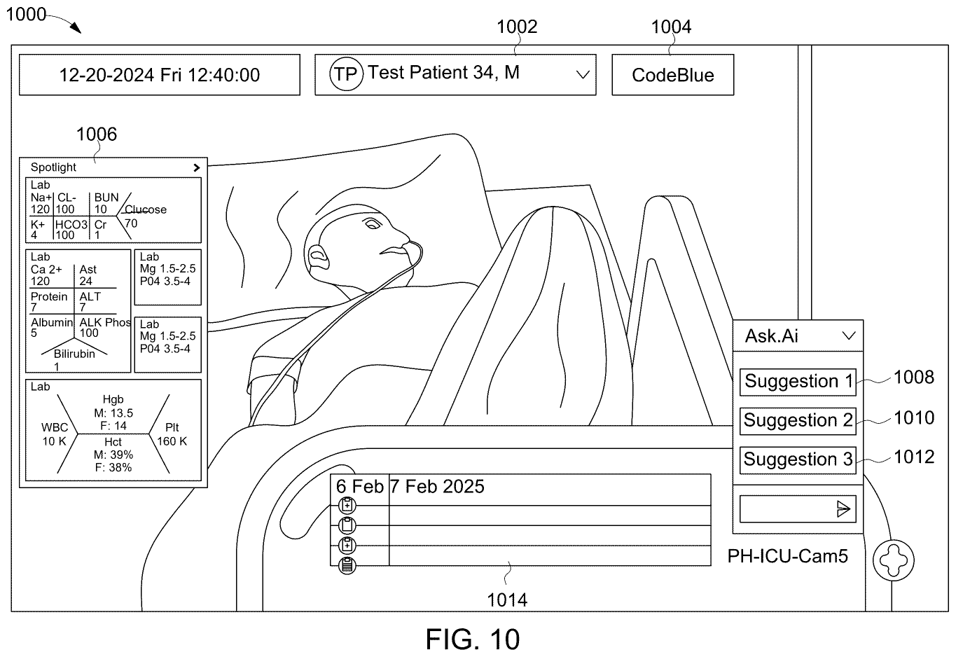

Referring now to FIG. 10, a GUI 1000 for real-time video analysis and clinical action execution via multi-agent orchestration is illustrated, in accordance with some embodiments of the present disclosure. FIG. 10 is explained in conjunction with FIG. 1-9. The GUI 1000 may display a real-time video feed of a patient in a hospital bed as the background, with the patient shown wearing medical equipment including nasal tubing. A patient information bar 1002 at the top centre of the GUI 1000 may display patient identification information (for example, name of the patient is “Abhinab Barman”) along with an alert button 1004. On the left side of the GUI 1000, a spotlight panel 1006 may display laboratory values and vital signs data organized in multiple sections showing values for parameters such as glucose, potassium, HCO3, creatinine, magnesium, phosphate, WBC count, haemoglobin, haematocrit, and platelet count. On the right side of the GUI 1000, an AI interaction interface may present a first suggestion 1008, a second suggestion 1010, and a third suggestion 1012 as selectable options for user interaction with the AI system. A timeline interface 1014 may appear at the bottom of the GUI 1000 showing a date indicator with various event markers and navigation controls. A text input field 1018 with a send button may be positioned below the suggestions in the AI interaction interface, allowing users to submit queries to the AI system.

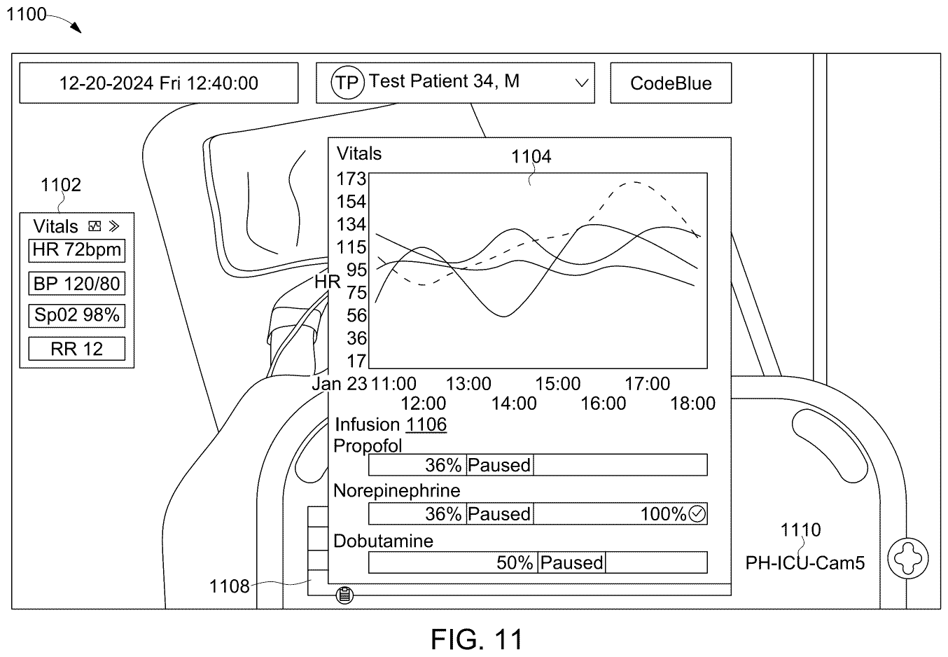

Referring now to FIG. 11, a GUI 1100 for real-time video analysis and clinical action execution via multi-agent orchestration is illustrated, in accordance with some embodiments of the present disclosure. FIG. 11 is explained in conjunction with FIG. 1-10. The GUI 1100 may display a real-time video feed of a patient in a hospital bed as the background, with the patient visible lying in the bed. A patient information bar may be shown at the top of the GUI 1100, displaying patient identification information including initials, name, age, and gender, along with an alert indicator. A vitals panel 1102 may be positioned on the left side of the GUI 1100, showing current patient vital signs including heart rate, blood pressure, SpO2, and respiratory rate. A vitals graph 1104 may be displayed in the center of the GUI 1100, showing multiple trend lines representing vital signs over time. An infusion panel 1106 may be shown below the vitals graph, displaying medication infusion status for various medications with progress indicators showing paused and active states. A timeline interface 1108 may be positioned at the bottom of the GUI 1100, allowing navigation through recorded video segments. A camera identifier 1110 may be displayed in the lower right portion of the GUI 1100, along with an AI interaction interface icon 1112.

As will be also appreciated, the above-described techniques may take the form of computer or controller implemented processes and apparatuses for practicing those processes. The disclosure can also be embodied in the form of computer program code containing instructions embodied in tangible media, such as floppy diskettes, solid state drives, CD-ROMs, hard drives, or any other computer-readable storage medium, wherein, when the computer program code is loaded into and executed by a computer or controller, the computer becomes an apparatus for practicing the invention. The disclosure may also be embodied in the form of computer program code or signal, for example, whether stored in a storage medium, loaded into and/or executed by a computer or controller, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

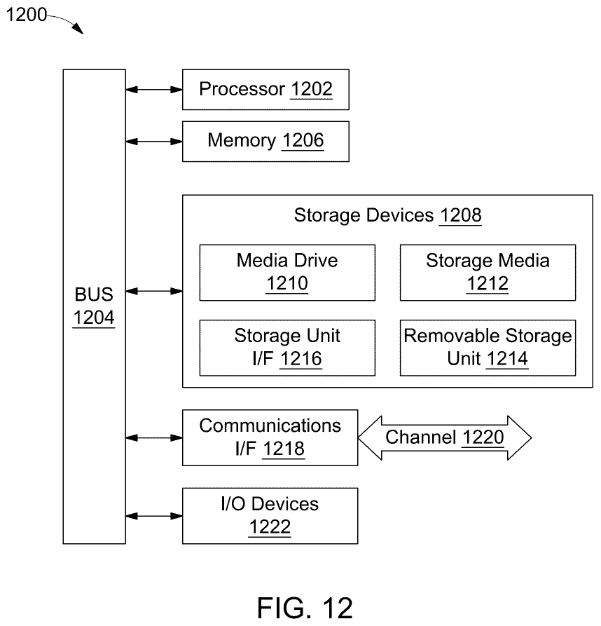

The disclosed methods and systems may be implemented on a conventional or a general-purpose computer system, such as a personal computer (PC) or server computer. Referring now to FIG. 12, an exemplary computing system 1200 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 1200 may represent, for example, a user device such as a desktop, a laptop, a mobile phone, personal entertainment device, DVR, and so on, or any other type of special or general-purpose computing device as may be desirable or appropriate for a given application or environment. The computing system 1200 may include one or more processors, such as a processor 1202 that may be implemented using a general or special purpose processing engine such as, for example, a microprocessor, microcontroller or other control logic. In this example, the processor 1202 is connected to a bus 1204 or other communication medium. In some embodiments, the processor 1202 may be an Artificial Intelligence (AI) processor, which may be implemented as a Tensor Processing Unit (TPU), or a graphical processor unit, or a custom programmable solution Field-Programmable Gate Array (FPGA).

The computing system 1200 may also include a memory 1206 (main memory), for example, Random Access Memory (RAM) or other dynamic memory, for storing information and instructions to be executed by the processor 1202. The memory 1206 also may be used for storing temporary variables or other intermediate information during execution of instructions to be executed by the processor 1202. The computing system 1200 may likewise include a read only memory (“ROM”) or other static storage device coupled to bus 1204 for storing static information and instructions for the processor 1202.

The computing system 1200 may also include storage devices 1208, which may include, for example, a media drive 1210 and a removable storage interface. The media drive 1210 may include a drive or other mechanism to support fixed or removable storage media, such as a hard disk drive, a floppy disk drive, a magnetic tape drive, an SD card port, a USB port, a micro-USB, an optical disk drive, a CD or DVD drive (R or RW), or other removable or fixed media drive. A storage media 1212 may include, for example, a hard disk, magnetic tape, flash drive, or other fixed or removable medium that is read by and written to by the media drive 1210. As these examples illustrate, the storage media 1212 may include a computer-readable storage medium having stored there in particular computer software or data.

In alternative embodiments, the storage devices 1208 may include other similar instrumentalities for allowing computer programs or other instructions or data to be loaded into the computing system 1200. Such instrumentalities may include, for example, a removable storage unit 1214 and a storage unit interface 1216, such as a program cartridge and cartridge interface, a removable memory (for example, a flash memory or other removable memory module) and memory slot, and other removable storage units and interfaces that allow software and data to be transferred from the removable storage unit 1214 to the computing system 1200.

The computing system 1200 may also include a communications interface 1218. The communications interface 1218 may be used to allow software and data to be transferred between the computing system 1200 and external devices. Examples of the communications interface 1218 may include a network interface (such as an Ethernet or other NIC card), a communications port (such as for example, a USB port, a micro-USB port), Near field Communication (NFC), etc. Software and data transferred via the communications interface 1218 are in the form of signals which may be electronic, electromagnetic, optical, or other signals capable of being received by the communications interface 1218. These signals are provided to the communications interface 1218 via a channel 1220. The channel 1220 may carry signals and may be implemented using a wireless medium, wire or cable, fiber optics, or other communications medium. Some examples of the channel 1220 may include a phone line, a cellular phone link, an RF link, a Bluetooth link, a network interface, a local or wide area network, and other communications channels.

The computing system 1200 may further include Input/Output (I/O) devices 1222. Examples may include, but are not limited to a display, keypad, microphone, audio speakers, vibrating motor, LED lights, etc. The I/O devices 1222 may receive input from a user and also display an output of the computation performed by the processor 1202. In this document, the terms “computer program product” and “computer-readable medium” may be used generally to refer to media such as, for example, the memory 1206, the storage devices 1208, the removable storage unit 1214, or signal(s) on the channel 1220. These and other forms of computer-readable media may be involved in providing one or more sequences of one or more instructions to the processor 1202 for execution. Such instructions, generally referred to as “computer program code” (which may be grouped in the form of computer programs or other groupings), when executed, enable the computing system 1200 to perform features or functions of embodiments of the present invention.

In an embodiment where the elements are implemented using software, the software may be stored in a computer-readable medium and loaded into the computing system 1200 using, for example, the removable storage unit 1214, the media drive 1210 or the communications interface 1218. The control logic (in this example, software instructions or computer program code), when executed by the processor 1202, causes the processor 1202 to perform the functions of the invention as described herein.

Various embodiments provide method and system for real-time video analysis and clinical action execution via multi-agent orchestration. The disclosed method and system may receive a real-time video of a patient from a camera. The real-time video may include a plurality of frames. Further, the disclosed method and system may assign, via an AI model, an index tag to each of the plurality of frames in the real-time video. The index tag may correspond to a textual description of an associated frame(s). Upon receiving a query for the patient, the disclosed method and system may retrieve, via the AI model, the plurality of frames and an associated plurality of index tags of the plurality of frames. The query is one of a user query received via a GUI or a system prompt. Further, the disclosed method and system may determine, via the AI model, one or more of a plurality of AI agents based on the user query, the plurality of frames, and the associated plurality of index tags. Each of the plurality of AI agents may be preconfigured for executing a task. Further, the disclosed method and system may generate, via the AI model, a combined response for the user query based on an agent response of each of the one or more of the plurality of AI agents.

Thus, the disclosed techniques try to overcome the problem for real-time video analysis and clinical action execution via multi-agent orchestration. The techniques may overcome the problem of delay in taking remedial actions in case of undesired vitals of the patient. The techniques may provide better care and supervision to the patient. The techniques may further incorporate easier access of the doctor through an AV interface. The techniques may further include easier user query resolution via an AI interface. The techniques may further keep track of improvement or deterioration of the health of the patient through the EMR data. Indexing of video frames ensures resource efficient and time-effective retrieval of relevant data from the databases. Additionally, the agentic framework allows efficient orchestration of various task-specific AI models to address the user queries.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The specification has a described method and system for real-time video analysis and clinical action execution via multi-agent orchestration. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, non-volatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A method for real-time video analysis and clinical action execution via multi-agent orchestration, the method comprising:
receiving, by a processor, a real-time video of a patient from a camera, wherein the real-time video comprises a plurality of frames;
assigning, by the processor via an Artificial Intelligence (AI) model, an index tag to each of the plurality of frames in the real-time video, wherein the index tag corresponds to a textual description of an associated frame(s);
upon receiving a query for the patient, retrieving, by the processor via the AI model, the plurality of frames and an associated plurality of index tags of the plurality of frames, wherein the query is one of a user query received via a Graphical User Interface (GUI) or a system prompt;
determining, by the processor via the AI model, one or more of a plurality of AI agents based on the user query, the plurality of frames, and the associated plurality of index tags, wherein each of the plurality of AI agents is preconfigured for executing a task; and
generating, by the processor via the AI model, a combined response for the user query based on an agent response of each of the one or more of the plurality of AI agents.

2. The method of claim 1, further comprising:
identifying, via a set of Computer Vision (CV) models, one or more objects around the patient in a frame of the real-time video;
for each object of the one or more objects,
determining, via the set of CV models, an area of coverage of the object in the frame; and
when the area of coverage is above a predefined threshold, retrieving, via the AI model, at least one of relevant patient data, device data, or environment data associated with the object.

3. The method of claim 1, wherein determining the one or more of the plurality of AI agents comprises determining an order of processing of the user query through the one or more of the plurality of AI agents.

4. The method of claim 3, wherein generating the combined response comprises sequentially processing the user query, the plurality of frames, and the associated plurality of index tags through the one or more of the plurality of AI agents in the order of processing to obtain the combined response.

5. The method of claim 1, wherein generating the combined response comprises:
for each AI agent of the one or more of the plurality of AI agents,
creating, via the AI agent, a prompt based on a set of agent instructions and at least one of the user query, the plurality of frames, or the associated plurality of index tags;
inputting, via the AI agent, the prompt to the AI model; and
generating, via the AI model, the agent response based on the prompt;
and
combining, via the AI model, the agent response for each of the one or more of the plurality of AI agents to obtain the combined response.

6. The method of claim 5, wherein the plurality of AI agents comprises at least one of a patient data retrieval agent, an EMR management agent, a camera control agent, and a communication agent.

7. The method of claim 6, wherein generating the agent response comprises, at least one of:
generating, via the patient data retrieval agent, a patient summary based on at least one of patient data retrieved from an EMR of the patient or medical domain data retrieved from a medical knowledge base;
executing, via the EMR management agent, an order action in the EMR through a first Application Programming Interface (API) based on the user query, wherein the order action corresponds to one of a new order addition, an order cancellation, or an order modification;
transmitting, via the camera control agent, a control instruction to the camera through a second API based on the user query for execution of a camera action to modify a current field of view (FoV) of the camera, wherein the camera action corresponds to one of a pan action, a tilt action, or a zoom action; or
executing, via the communication agent, a communication action on the GUI through a third API based on the user query, wherein the communication action is one of a message transmission or a call initiation.

8. The method of claim 1, further comprising rendering, via the GUI, the real-time video of the patient and the combined response to the user query, wherein the combined response is overlaid on the real-time video of the patient in the GUI or is sent as a user alert.

9. A system for real-time video analysis and clinical action execution via multi-agent orchestration, the system comprising:
a processor; and
a memory communicatively coupled to the processor, wherein the memory stores processor instructions, which when executed by the processor, cause the processor to:
receive a real-time video of a patient from a camera, wherein the real-time video comprises a plurality of frames;
assign, via an Artificial Intelligence (AI) model, an index tag to each of the plurality of frames in the real-time video, wherein the index tag corresponds to a textual description of an associated frames;
upon receiving a query for the patient, retrieve, via the AI model, the plurality of frames and an associated plurality of index tags of the plurality of frames, wherein the query is one of a user query received via a Graphical User Interface (GUI) or a system prompt;
determine, via the AI model, one or more of a plurality of AI agents based on the user query, the plurality of frames, and the associated plurality of index tags, wherein each of the plurality of AI agents is preconfigured for executing a task; and
generate, via the AI model, a combined response for the user query based on an agent response of each of the one or more of the plurality of AI agents.

10. The system of claim 9, wherein the processor instructions, on execution, further cause the processor to:
identify, via a set of Computer Vision (CV) models, one or more objects around the patient in a frame of the real-time video;
for each object of the one or more objects,
determine, via the set of CV models, an area of coverage of the object in the frame; and
when the area of coverage is above a predefined threshold, retrieve, via the AI model, at least one of relevant patient data, device data, or environment data associated with the object.

11. The system of claim 9, wherein to determine the one or more of the plurality of AI agents, the processor instructions, on execution, cause the processor to determine an order of processing of the user query through the one or more of the plurality of AI agents.

12. The system of claim 11, wherein to generate the combined response, the processor instructions, on execution, cause the processor to sequentially process the user query, the plurality of frames, and the associated plurality of index tags through the one or more of the plurality of AI agents in the order of processing to obtain the combined response.

13. The system of claim 9, wherein to generate the combined response, the processor instructions, on execution, cause the processor to:
for each AI agent of the one or more of the plurality of AI agents,
create, via the AI agent, a prompt based on a set of agent instructions and at least one of the user query, the plurality of frames, or the associated plurality of index tags;
input, via the AI agent, the prompt to the AI model; and
generate, via the AI model, the agent response based on the prompt; and
combine, via the AI model, the agent response for each of the one or more of the plurality of AI agents to obtain the combined response.

14. The system of claim 13, wherein the plurality of AI agents comprises at least one of a patient data retrieval agent, an EMR management agent, a camera control agent, and a communication agent.

15. The system of claim 14, wherein to generate the agent response, the processor instructions, on execution, cause the processor to, at least one of:
generate, via the patient data retrieval agent, a patient summary based on at least one of patient data retrieved from an EMR of the patient or medical domain data retrieved from a medical knowledge base;
execute, via the EMR management agent, an order action in the EMR through a first Application Programming Interface (API) based on the user query, wherein the order action corresponds to one of a new order addition, an order cancellation, or an order modification;
transmit, via the camera control agent, a control instruction to the camera through a second API based on the user query for execution of a camera action to modify a current field of view (FoV) of the camera, wherein the camera action corresponds to one of a pan action, a tilt action, or a zoom action; or
execute, via the communication agent, a communication action on the GUI through a third API based on the user query, wherein the communication action is one of a message transmission or a call initiation.

16. The system of claim 9, wherein the processor instructions, on execution, cause the processor to render, via the GUI, the real-time video of the patient and the combined response to the user query, wherein the combined response is overlaid on the real-time video of the patient in the GUI or is sent as user alert.

17. A non-transitory computer-readable medium storing computer-executable instructions for real-time video analysis and clinical action execution via multi-agent orchestration, the computer-executable instructions configured for:
receiving a real-time video of a patient from a camera, wherein the real-time video comprises a plurality of frames;
assigning, via an Artificial Intelligence (AI) model, an index tag to each of the plurality of frames in the real-time video, wherein the index tag corresponds to a textual description of an associated frame(s);
upon receiving a query for the patient, retrieving, via the AI model, the plurality of frames and an associated plurality of index tags of the plurality of frames, wherein the query is one of a user query received via a Graphical User Interface (GUI) or a system prompt;
determining, via the AI model, one or more of a plurality of AI agents based on the user query, the plurality of frames, and the associated plurality of index tags, wherein each of the plurality of AI agents is preconfigured for executing a task; and
generating, via the AI model, a combined response for the user query based on an agent response of each of the one or more of the plurality of AI agents.

18. The non-transitory computer-readable medium of claim 17, wherein the computer-executable instructions are further configured for:
identifying, via a set of Computer Vision (CV) models, one or more objects around the patient in a frame of the real-time video;
for each object of the one or more objects,
determining, via the set of CV models, an area of coverage of the object in the frame; and
when the area of coverage is above a predefined threshold, retrieving, via the AI model, at least one of relevant patient data, device data, or environment data associated with the object.

19. The non-transitory computer-readable medium of claim 18, wherein for determining the one or more of the plurality of AI agents, the computer-executable instructions are configured for determining an order of processing of the user query through the one or more of the plurality of AI agents.

20. The non-transitory computer-readable medium of claim 19, wherein for generating the combined response, the computer-executable instructions are configured for sequentially processing the user query, the plurality of frames, and the associated plurality of index tags through the one or more of the plurality of AI agents in the order of processing to obtain the combined response.
